(19) United States
(12) Patent Application Publication
(10) Pub. No.: US 2018/0085009 A1
(43) Pub. Date: Mar. 29, 2018

(54) METHOD AND SYSTEM FOR DETECTING USER HEART RATE USING LIVE CAMERA FEED

(71) Applicant: OCR Labs Pty Ltd, Bella Vista (AU)

(72) Inventors: Daniel Aiello, St. Ives (AU); Matthew Adams, Kings Langley (AU)

(21) Appl. No.: 15/717,165

(22) Filed: Sep. 27, 2017

(30) Foreign Application Priority Data
Sep. 27, 2016 (AU) 2016903901

(51) Int. Cl. A61B 5/0205; A61B 5/1171; A61B 5/00; A61B 5/11

(52) U.S. Cl. CPC A61B 5/0205; A61B 5/1176; A61B 5/02427; A61B 5/1103; A61B 5/6898; A61B 5/7278

(57) ABSTRACT

A system and method for detecting a heart rate of a user using a portable electronic device is provided. The system includes a first camera mounted to the portable electronic device for taking a series of photographs of the user, a memory for storing the series of photographs including the RGB tone colour details of each pixel for each photograph, and a processor for removing the red and blue tones from each image and for measuring the variation in green tone between each image thereby detecting the heart rate of the user.

### CROSS-REFERENCE TO RELATED APPLICATION

This application claims priority to Australian Application No. 2016903901 which was filed on Sep. 27, 2016, the contents of which are incorporated by reference.

### FIELD

The present invention relates to fraud detection systems and in particular to automated facial recognition systems.

The invention has been developed primarily for use with a mobile phone camera and will be described hereinafter with reference to this application. However, it will be appreciated that the invention is not limited to this particular field of use and can be used in other applications and with other types of cameras, for example, as those installed on tablet computers, laptops and webcams.

### BACKGROUND

In this specification unless the contrary is expressly stated, where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

Throughout the specification and claims which follow, unless the context requires otherwise, the word “comprise”, and other variations such as “comprises” and “comprising”, will be understood to imply the inclusion of a stated integer or step or group of integers or steps but not the exclusion of any other integer or step or group of integers of steps.

Many service providers require clients' to be formally identified before being able to provide them with services. Identification of a client is usually performed using a government issued document such as a driver's licence, birth certificate, proof of age card, passport, student card or healthcare card. In some instances a non-government document or card may also be used such as a credit card, bank card, student ID card or private healthcare card.

In the prior art, identification documents have been validated and proofed by a human operator. Human operators are also trained on fraud detection and look out for fraudulent documents. With the expansion of mobile phone use it is now possible to collect copies of identification documents using the mobile phone camera and an application or app installed on the phone or running on the web.

However, digital copies of identification documents cannot always collect and capture all information stored on identification documents such as holograms and watermarks and as such there is an increased risk of fraud when capturing these documents electronically. In addition not having to present original documents also open the opportunity for fraudulent and counterfeit documents.

### SUMMARY

Preferred embodiments of the present invention overcome or ameliorate at least one of the disadvantages of the prior art, or to provide a useful alternative.

According to a first aspect of the invention there is provided a system for detecting a heart rate of a user using a portable electronic device, the system including: a first camera mounted to the portable electronic device for taking a series of photographs of the user; a memory for storing the series of photographs including the RGB tone colour details of each pixel for each photograph; a processor for removing the red and blue tones from each image and for measuring the variation in green tone between each image thereby detecting the heart rate of the user. Preferably the portable electronic device includes a second camera for taking a second heart rate from the user's finger. Preferably the processor measures the variance between the heart rate detected at the first camera and the second camera. Preferably the processor detects movement detected from the cardiac pulse cause by blood flow in the user. Preferably the processor further measures blinking and facial movements to determine whether the user is moving. Preferably the portable electronic device is a mobile phone or tablet computer.

According to a second aspect of the invention there is provided method for detecting a heart rate of a user using a portable electronic device, the method including: providing a portable electronic device having a first camera for taking a series of images of the user; providing a memory for storing the series of images including the RGB tone colour details of each pixel for each photograph; providing a processor for analysing series of images to determine whether the user has heart rate. Preferably the method includes the steps of removing: the red and blue tones from each image; measuring the variation in green tone between each image; and determining when there is variation in green tones that the user has heart rate.

Another preferred embodiment of the invention provides a system for capturing and validating an identification document having a photograph of the user. The system then takes a photograph of the user with a mobile phone camera. The system then uses facial recognition to determine whether the photograph taken using the mobile phone camera matches a photo on the identification document. In this way the system can determine whether the photograph on the identification document and the person whose photograph is taken by the mobile phone are the same person or a different person.

Preferably the mobile phone camera captures a continuous series of photographs of the user or takes a video of the user. The series of photos is analysed to ensure that the user is not simply holding up a photograph of the person in the identification document in an attempt to commit fraud.

Preferably the series of photographs is analysed for blink detection and for heart rate detection to verify that the photographs are of a real and live person. Preferably, additional level of security is enabled for higher security environments in which the both the front facing mobile phone camera and the back facing mobile phone camera are used to detect the heart rate of the user. In this scenario, the front facing camera detects the heart rate of the user's face and the user places a finger, preferably their index finger, on the back camera. If the heart rates are the same then the risk of fraud is reduced. If the heart rates are different then there is a high risk of some fraudulent activity. This prevents the user from pointing the camera on someone else's face or pointing the camera to a high definition video stream.

Preferably the detection system is implemented in a Software Development Kit (SDK) using an algorithm or different algorithms that can be tailored to different environments and different user skin colours.

Preferably, the system can detect a user's emotion through correlation of the heart rate with predetermined heart rate patterns. For example, excitement increases the heart rate so if the user's heart rate is increased above the normal range they may be excited. Further embodiments can be applied to the medical field and for medical implementations.

Preferably, the system can be used for proofing of reality and confirmation of movement and real person detection in the area of facial recognition in access login allowing user's to use their face to login to computers instead of a password.

Embodiments of the invention can be used to prevent fraud through making the use of stolen or counterfeit documents more difficult. This is done as users cannot user a printed photograph of another user as a mask, cannot superimpose a photograph on an identification document and cannot print and use a 3D head of another person in an attempt to overcome some verification checks.

### BRIEF DESCRIPTION OF THE DRAWINGS

A preferred embodiment of the invention will now be described, by way of example only, with reference to the accompanying drawings in which:

FIG. 1 shows a diagram of a mobile phone capturing an identification document according to the preferred embodiment of the invention;

FIG. 2 shows a diagram of a user using a phone according to the preferred embodiment of the invention;

FIG. 3 shows diagram of a user blinking according to the preferred embodiment of the invention;

FIG. 4 shows a diagram of a mobile phone capturing the heart rate of a user according to the preferred embodiment of the invention; and

FIG. 5 shows a diagram of a mobile phone capturing the heart rate of a user according to the preferred embodiment of the invention.

### DETAILED DESCRIPTION OF EMBODIMENTS

The preferred embodiment of the invention provides a system for capturing and validating an identification document having a photograph of the user. The system then takes a photograph of the user with a mobile phone camera. The system then uses facial recognition to determine whether the photograph taken using the mobile phone camera matches a photo on the identification document. In this way the system can determine whether the photograph on the identification document and the person whose photograph is taken by the mobile phone are the same person or a different person.

The mobile phone camera captures a continuous series of photographs of the user or takes a video of the user. The series of photos is analysed to ensure that the user is not simply holding up a photograph of the person in the identification document in an attempt to commit fraud. The series of photographs is analysed for blink detection and for heart rate detection to verify that the photographs are of a real and live person. An additional level of security is enabled for higher security environments in which the both the front facing mobile phone camera and the back facing mobile phone camera are used to detect the heart rate of the user. In this scenario, the front facing camera detects the heart rate of the user's face and the user places a finger, preferably their index finger, on the back camera. If the heart rates are the same then the risk of fraud is reduced. If the heart rates are different then there is a high risk of some fraudulent activity. This prevents the user from pointing the camera on someone else's face or pointing the camera to a high definition video stream.

The detection system is implemented in a Software Development Kit (SDK) using an algorithm or different algorithms that can be tailored to different environments and different user skin colours.

The system can detect a user's emotion through correlation of the heart rate with predetermined heart rate patterns. For example, excitement increases the heart rate so if the user's heart rate is increased above the normal range they may be excited. Further embodiments can be applied to the medical field and for medical implementations.

The system can be used for proofing of reality and confirmation of movement and real person detection in the area of facial recognition in access login allowing user's to use their face to login to computers instead of a password.

Embodiments of the invention can be used to prevent fraud through making the use of stolen or counterfeit documents more difficult. This is done as users cannot user a printed photograph of another user as a mask, cannot superimpose a photograph on an identification document and cannot print and use a 3D head of another person in an attempt to overcome some verification checks.

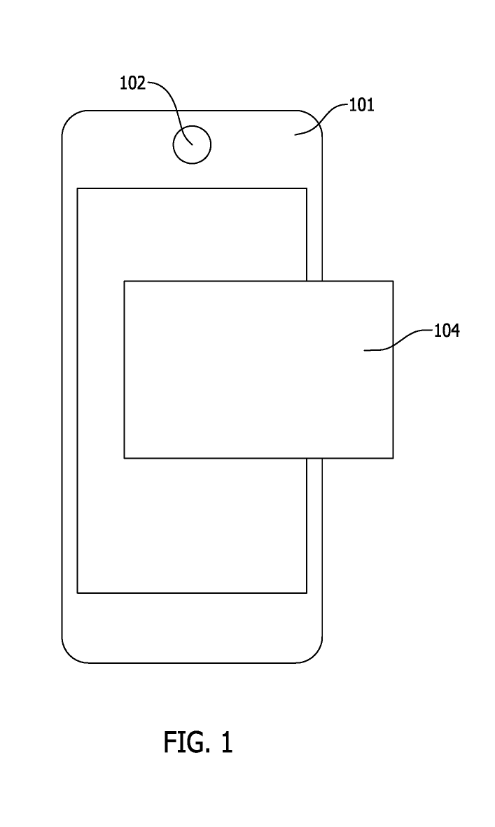

Referring to FIG. 1, there is a shown a mobile phone 101 having a front camera 102 and back camera 103. A user 105 opens the App and captures information from an identity document 104 with either the front or back camera. Alternatively the user manually enters in credential information to bring up a previously stored virtual identity card (virtually stored ID such as a virtual drivers license or a database stores ID).

When capturing information from a physical document, the Information Document (ID) form is recognised from a number of predetermined parameters and facial recognition is performed on the ID to identify a face on or within a predefined fixed area of the card.

The facial information from the ID document is then extracted and stored within the App and can be transferred to a remote server as necessary.

When retrieving information from a virtual ID, the user enters their identification number and proceeds to the next step in the App. The App verifies the user's ID and this is verified with the virtual ID servers. The App then proceeds to capture the user's face 106.

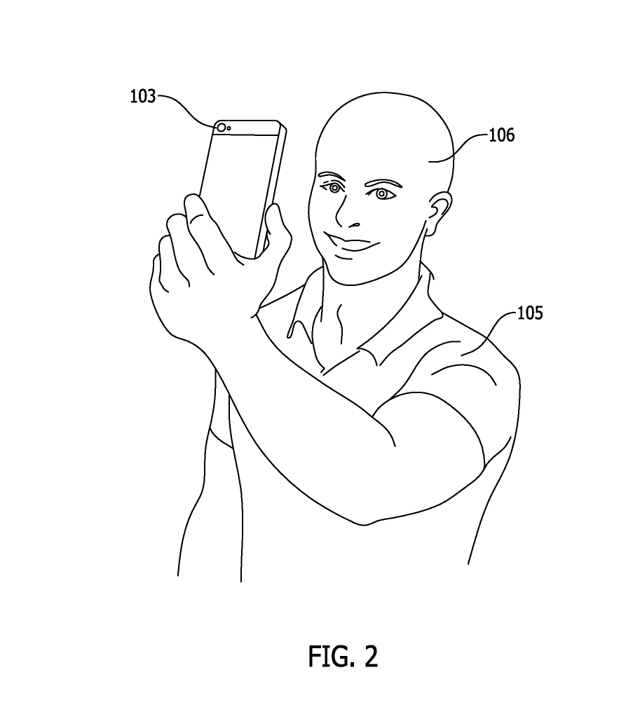

Referring to FIG. 2 the user then proceeds to take a photo of their face with the phone's camera. The camera will identify the face and will also look for signs that the person a real live person through characteristics such as looking for user blinks 107 and movement of the users face and head.

The lighting in the environment is checked to ensure that there is no over saturation of light or darkness as adjustments to the camera are made if these are detected. Once the camera activates it will start capture a series of photos passing this in real time for processing for heart rate analysis. The analysis is done on the mobile phone but can be processed remotely in some instance.

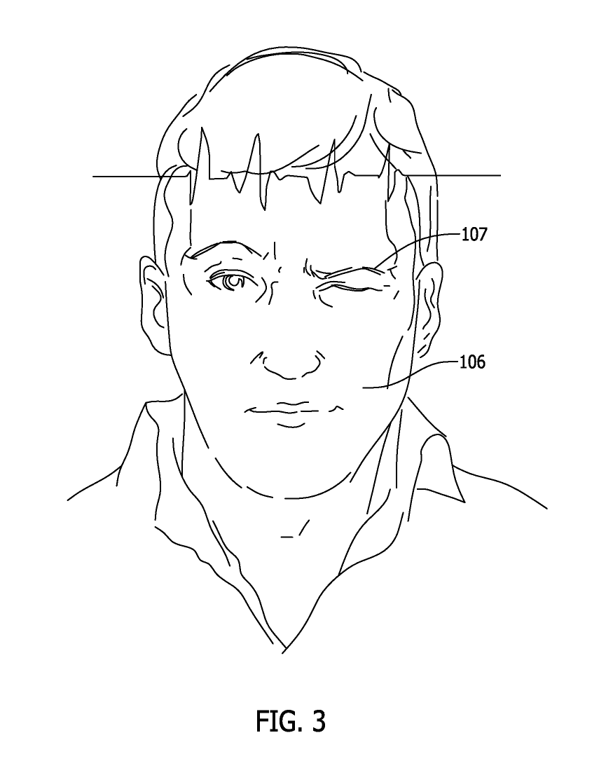

Referring to FIG. 3, while capturing each frame the colour changes at each pixel on the face and forehead. By removing the red and blue colours, this leaves the green tones of the image. These variations of green are measured to the amount of how much the green tone increases and decreases providing a determinable heart rate and therefore detecting “liveness” or determining that the subject is alive. Image processing is done using RGB processing and Independent Component Analysis (ICA) as detailed below. In the preferred embodiment processing of the image is done on the mobile phone.

The system also detects movement from the cardiac pulse caused by blood flow which makes the head move subtly. The system utilises video amplification that is able to detect these subtle movements frame by frame wherein each movement correlates to a heartbeat. This can be used along with blinking and facial movements to accurately determine the face being of reality an alive.

If phone determines the subject is live then the App shows that verification was successful and the Application can then continue to the next stage or step. If there is a fail then the system can either prompt the user that they need to use a real face (not a picture) or the system can silently flag the failure in the background.

The system uses RGB and ICA (Independent Component Analysis) to determine the heart rate of the user or to validate a heart rate of the user to determine confirm they are a live user. It also uses the raw green signal used in the RGB from the video. The system first Identifies the PPG (photoplethysmogram) signal which results in having to separate the face from the background. This is done by deploying a facial recognition process to track the face and capture a video stream which contains a series of images. This allows the system to accurately check each pixel movement from frame to frame and facial skin pixels.

$${y}_{i , j}^{′} = {y}_{i , j} - \frac{1}{2 n + 1} {\sum }_{k = - n}^{n} {y}_{i + k , j}$$

The system uses a boundary in the shape of a face where crops the users face from the video stream. This is referred to as an ROI (Region of Interest). For each frame, the average value of all pixels in the ROI is calculated. This yields yi,j as the average value of colour j ∈ {red, green, blue} in the ROI of the i^{th} frame. For an entire movie and a specific colour, j, this is a signal yj. where yi′,j is the detrended version of yi,j·2n+1 (moving average). Based on this PPG signal and by combining the colour change signals obtained from different regions of the face the system calculates a weighted average. If this is detected then it is understood that there is a heart rate.

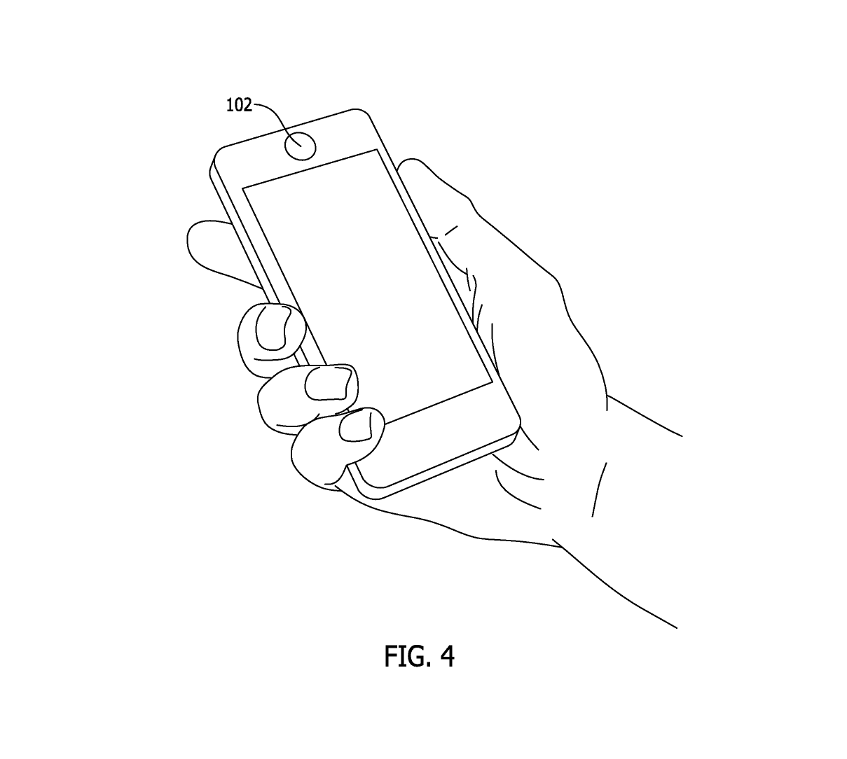

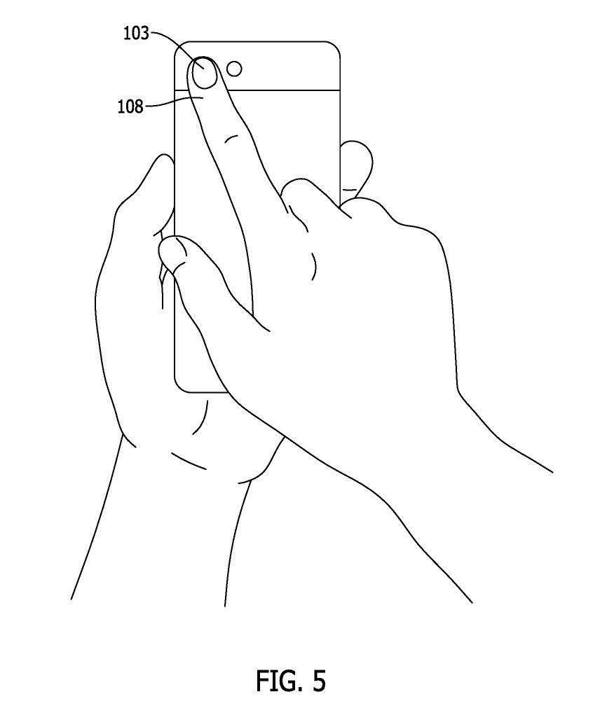

Referring now to FIGS. 4 and 5, in higher security environments a higher level of security heard rate monitoring is enabled. In this environment there is a comparison function that measures the heart rate of the user twice. The first measure if through the front camera 102 and is a measurement of the heart rate through the user's face 104. The second measurement is via the index finger 108 being placed on the rear camera of the phone that measures the heart rate of the user's finger. These measurements are then compared to determine the risk of fraudulent activity. Analysis of the images from the user's finger is done by using RGB processing and Independent Component Analysis as described above.

As would be understood features of different embodiments can be combined as required to suite the particular application.

Embodiments of the invention can be performed using any mobile phone or tablet computer. These include by way of non limiting example Apple™ iPhone, iPad, Android phones and tablets, Samsung smart phones and tablets.

There are hundreds of available computer languages that may be used to implement embodiments of the invention, among the more common being Ada; Algol; APL; awk; Basic; C; C++; Cobol; Delphi; Eiffel; Euphoria; Forth; Fortran; HTML; Icon; Java; Javascript; Lisp; Logo; Mathematica; MatLab; Miranda; Modula-2; Oberon; Pascal; Perl; PL/I; Prolog; Python; Rexx; SAS; Scheme; sed; Simula; Smalltalk; Snobol; SQL; Visual Basic; Visual C++; and XML.

Any commercial processor may be used to implement the embodiments of the invention either as a single processor, serial or parallel set of processors in the system. Examples of commercial processors include, but are not limited to Merced™, Pentium™, Pentium II™, Xeon™, Celeron™, Pentium Pro™, Efficeon™, Athlon, AMD, Intel Core™ i3, i5, i7 and the like.

Display screens may be segment display screen, analogue display screens, digital display screens, CRTs, LED screens, Plasma screens, liquid crystal diode screens, and the like.

Although the invention has been described with reference to specific examples, it will be appreciated by those skilled in the art that the invention may be embodied in many other forms.

What is claimed is:

1. A system for detecting a heart rate of a user using a portable electronic device, the system including:
a first camera mounted to the portable electronic device for taking a series of photographs of the user;
a memory for storing the series of photographs including the RGB tone colour details of each pixel for each photograph;
a processor for removing the red and blue tones from each image and for measuring the variation in green tone between each image thereby detecting the heart rate of the user.

2. A system according to claim 1 wherein the portable electronic device includes a second camera for taking a second heart rate from the user's finger.

3. A system according to claim 2 wherein the processor measures the variance between the heart rate detected at the first camera and the second camera.

4. A system according to claim 3 wherein the processor detects movement detected from the cardiac pulse cause by blood flow in the user.

5. A system according to claim 4 wherein the processor further measures blinking and facial movements to determine whether the user is moving.

6. A system according to claim 5 wherein the portable electronic device is a mobile phone or tablet computer.

7. A method for detecting a heart rate of a user using a portable electronic device, the method including:
providing a portable electronic device having a first camera for taking a series of images of the user;
providing a memory for storing the series of images including the RGB tone colour details of each pixel for each photograph;
providing a processor for analysing series of images to determine whether the user has heart rate.

8. The method of claim 7 including the steps of removing: the red and blue tones from each image; measuring the variation in green tone between each image; and determining when there is variation in green tones that the user has heart rate.
